(19) United States
(12) Patent Application Publication
(10) Pub. No.: US 2014/0087962 A1
(43) Pub. Date: Mar. 27, 2014

(54) Identification of Linkage Using Multiplex Digital PCR

(71) Applicant: David N. Keys, Alameda, CA (US)

(72) Inventor: David N. Keys, Alameda, CA (US)

(73) Assignee: LIFE TECHNOLOGIES CORPORATION, Carlsbad, CA (US)

(21) Appl. No.: 14/006,560

(22) PCT Filed: Mar. 22, 2012
(86) PCT No.: PCT/US12/30183

§ 371 Date: Nov. 22, 2013

Related U.S. Application Data
(60) Provisional application No. 61/466,272, filed on Mar. 22, 2011.

(51) Int. Cl. C12Q 1/68

(52) U.S. Cl. CPC C12Q 1/6858
USPC 506/9

(57) ABSTRACT

This specification generally relates to methods of detecting the linkage between two or more targets in a sample using digital multiplex PCR. A method of identifying physical linkage between two or more nucleic acid targets in a sample is provided. The method includes diluting the sample via limiting dilution and aliquoting the diluted sample into wells. The method further includes performing multiplex PCR in each chamber, where a first dye is used for the first target and a second dye is used for the second target. The method includes identifying the presence of the first and second dyes in the wells, wherein a non-random distribution of the dyes identifies the targets as linked.

### CROSS-REFERENCE TO RELATED APPLICATIONS

This application is a National Stage entry from PCT Application no. PCT/US2012/030183 filed Mar. 22, 2012, which claims a benefit under 35 USC §119(e) from U.S. Provisional Application No. 61/466,272 filed Mar. 22, 2011, both of which are incorporated herein by reference.

### FIELD

This specification generally relates to methods of identifying linkage using multiplex digital PCR techniques.

### BACKGROUND

Digital PCR (dPCR) was originally developed to overcome some of the difficulties of conventional PCR. With dPCR, a sample is partitioned so that individual nucleic acid molecules within the sample are localized and concentrated within many separate regions. The partitioning of the sample allows one to count the molecules by estimating according to Poisson Distribution. As a result, each part will contain “0” or“1” molecules, or a negative or positive reaction, respectively. After PCR amplification, nucleic acids may be quantified by counting the regions that contain PCR end-product (positive reactions). In conventional PCR, the starting copy number is proportional to the number of PCR amplification cycles. dPCR, however, is not dependent on the number of amplification cycles to determine the initial sample amount, eliminating the reliance on uncertain exponential data to quantify target nucleic acids and providing absolute quantification.

Linkage studies using classical genetics are in general long and drawn out and time consuming. They do not result in high resolution answers. fluorescence in situ hybridization (FISH) is a molecular cytogenetic technique that can detect chromosomal abnormalities that cannot be appreciated by standard chromosomal analysis (e.g., microdeletion syndromes) or when mitotic cells are not available for chromosomal analysis (e.g., X/Y FISH for cross-sex transplants). Briefly, metaphase chromosomes or interphase nuclei are denatured on the slide, as is the fluorescently labeled DNA probe. The probe and the chromosomes/nuclei are then hybridized, slides are washed, counterstained and analyzed by fluorescent microscopy. There are a number of different types of FISH probes including unique sequence probes (e.g., microdeletion syndromes), whole chromosome painting probes, repetitive probes (e.g., centromeric alpha satellite probes, subtelomeric probes), gene fusion probes (e.g., BCR/ABL in t(9;22) in CML and ALL) and break apart probes (e.g., MLL in 11q23 rearrangements in ALL and AML). While FISH can successfully be used to successfully identify translocations, the identification of the translocation site is not at high resolution.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which, in and of themselves, may also be inventions.

### SUMMARY

Some aspects of the invention are methods of identifying physical linkage between two or more nucleic acid targets in a sample, by diluting the sample via limiting dilution; aliquoting the diluted sample into wells; performing multiplex PCR in each chamber, wherein a first dye is used for the first target and a second dye is used for the second target; and identifying the presence of the first and second dyes in the wells, wherein a non-random distribution of the dyes identifies the targets as linked. In some embodiments, the dye is attached to a probe. In some embodiments, the dye is attached to a primer. In some embodiments, the multiplex PCR employs FRET, TaqMan®, Molecular Beacon, Amplifluor®, Scorpion™, Plexor™, or BHQplus™. In some embodiments, the non-random distribution is determined by Student's T-test, F-test, Chi-square test, Fisher-test, ANOVA test, or multiple comparison ANOVA test. In some embodiments, the non-random distribution is determined by a p-value of <0.05. In some embodiments, the targets are chosen from: SNPs, InDels, mutations, translocations, inversions, duplications, deletions, ring chromosomes, isochromosomes, splice regions, microsatellites, mature microRNAs, pri-microRNAs, pre-microRNAs, non-coding RNAs, mRNAs primary transcripts, genomic loci, alleles, multi-RNA complexes, splice variants, transposons, ribozymes, bacterial genes, viral nucleic acids, ribosomal RNAs, viral insertion sites, vector insertion sites, hypervariable regions, mitochondrial DNA, highly polymorphic regions, MHC regions, and/or MHC gene products. In some embodiments, one or more of the targets is polymorphic. In some embodiments, the method also includes preamplification of the sample before diluting the sample. In some embodiments, the method also includes determining the original linkage distance of the two targets. In some embodiments, the method also includes at least a third target and a third dye. In some embodiments, the method also includes at least four or more targets and four or more different dyes. In some embodiments, the dye is a fluorescent dye or a quantum dot. In some embodiments, the dye is chosen from: FAM, TET, HEX, Cy3, TMR, ROX, Texas red, LC red 640, Cy5, VIC, SYBR, LC red 705, JUN, ABY, TED, TZA, and SID. In some embodiments, the method is used to determine the linkage between one or more alleles of a polymorphic site. In some embodiments, the method is used to determine the linkage distance between one or more alleles of a polymorphic site. In some embodiments, the method is used to determine haplotype or haplogroup. In some embodiments, the method is used for DNA profiling, DNA testing, DNA typing genetic fingerprinting, ancestry analysis, paternity testing, or maternity testing. In some embodiments, the method is used for disease association, disease risk assessment, patient stratification, drug metabolism analysis or diagnosis. In some embodiments, the amplification product is identified by sequencing, partial sequencing or hybridization.

Other aspects are methods for detecting the physical linkage between two or more nucleic acid targets in a sample, by, for example, diluting the sample into mother wells to a limiting dilution; preamplifying the diluted sample in the wells using multiplex PCR without a dye; aliquoting the product of each mother well into multiple daughter wells; determining the presence of a first target using PCR with a dye; determining the presence of a second target using PCR with a dye; and identifying whether the first and second target are linked by identifying mother wells which generated a nonrandom distribution of the first target and second target in its daughter wells. In some embodiments, the PCR in the daughter wells uses primers having the same sequence as the primers used in the mother wells. In some embodiments, the PCR in the daughter wells is via nested PCR. In some embodiments, the dye is attached to a probe. In some embodiments, the dye is attached to a primer. In some embodiments, the multiplex PCR employs FRET, TaqMan®, Molecular Beacon, Amplifluor®, Scorpion™ Plexor™, or BHQplus™. In some embodiments, the non-random distribution is determined by Student's T-test, F-test, Chi-square test, Fisher-test, ANOVA test, or multiple comparison ANOVA test. In some embodiments, the non-random distribution is determined by a p-value of <0.05. In some embodiments, the targets are chosen from: SNPs, InDels, mutations, translocations, inversions, duplications, deletions, ring chromosomes, isochromosomes, splice regions, microsatellites, mature microRNAs, pri-microRNAs, pre-microRNAs, non-coding RNAs, mRNAs primary transcripts, genomic loci, alleles, multi-RNA complexes, splice variants, transposons, ribozymes, bacterial genes, viral nucleic acids, ribosomal RNAs, viral insertion sites, vector insertion sites, hypervariable regions, mitochondrial DNA, highly polymorphic regions, MHC regions, and MHC gene products. In some embodiments, one or more of the targets is polymorphic. In some embodiments, the method also includes determining the original linkage distance of the two targets. In some embodiments, the dye is a fluorescent dye or a quantum dot. In some embodiments, the dye is chosen from: FAM, TET, HEX, Cy3, TMR, ROX, Texas red, LC red 640, Cy5, VIC, SYBR, LC red 705, JUN, ABY, TED, TZA, and SID. In some embodiments, the method also includes at least a third target. In some embodiments, the method also includes at least four or more targets and four or more different dyes. In some embodiments, the method also includes 10,000 or more targets. In some embodiments, the method is used to determine the linkage between one or more alleles of a polymorphic site. In some embodiments, the method is used to determine the linkage distance between one or more alleles of a polymorphic site. In some embodiments, the method is used to determine haplotype or haplogroup. In some embodiments, the method is used for DNA profiling, DNA testing, DNA typing genetic fingerprinting, ancestry analysis, paternity testing, or maternity testing. In some embodiments, the method is used for disease association, disease risk assessment, patient stratification, drug metabolism analysis or diagnosis. In some embodiments, the amplification product is identified by sequencing, partial sequencing or hybridization.

Other aspects are methods for detecting the physical linkage between two or more nucleic acid targets in a sample, by diluting the sample into mother wells to a limiting dilution; preamplifying the diluted sample in the wells using singleplex or multiplex PCR without a dye with a first and second primer; aliquoting the product of each mother well into multiple daughter wells; determining the presence of a first target in the daughter wells using nested PCR with a dye, said nested PCR using a third and fourth primer which are nested within the first and second primers; determining the presence of a second target in the daughter wells using nested PCR with a dye, said nested PCR using a fifth and sixth primer which are nested within the first and second primers; and identifying whether the first and second target are linked by identifying mother wells which generated a nonrandom distribution of the first target and second target in its daughter wells. In some embodiments, the dye is attached to a probe. In some embodiments, the dye is attached to a primer. In some embodiments, the multiplex PCR employs FRET, TaqMan®, Molecular Beacon, Amplifluor®, Scorpion™, Plexor™, or BHQplus™. In some embodiments, the non-random distribution is by Student's T-test, F-test, Chi-square test, Fisher-test, ANOVA test, or multiple comparison ANOVA test. In some embodiments, the non-random distribution has a p-value of <0.05. In some embodiments, the targets are chosen from: SNPs, InDels, mutations, translocations, splice regions, microsatellites, microRNAs, mRNAs, genomic loci, alleles, multi-RNA complexes, splice variants, transposons, ribozymes, microRNAs, bacterial genes, viral nucleic acids and ribosomal RNAs. In some embodiments, one or more of the targets is polymorphic. In some embodiments, the method also includes determining the original linkage distance of the two targets. In some embodiments, the dye is a fluorescent dye or a quantum dot. In some embodiments, the dye is chosen from: FAM, TET, HEX, Cy3, TMR, ROX, Texas red, LC red 640, Cy5, VIC, SYBR, LC red 705, JUN, ABY, TED, TZA, and SID. In some embodiments, the method also includes at least a third target. In some embodiments, the method also includes at least four or more targets and four or more different dyes. In some embodiments, the method also includes 10,000 or more targets. In some embodiments, the method is used to determine the linkage between one or more alleles of a polymorphic site. In some embodiments, the method is used to determine the linkage distance between one or more alleles of a polymorphic site. In some embodiments, the method is used to determine haplotype or haplogroup. In some embodiments, the method is used for DNA profiling, DNA testing, DNA typing genetic fingerprinting, ancestry analysis, paternity testing, or maternity testing. In some embodiments, the method is used for disease association, disease risk assessment, patient stratification, drug metabolism analysis or diagnosis. In some embodiments, the amplification product is identified by sequencing, partial sequencing or hybridization.

Any of the above embodiments may be used alone or together with one another in any combination. Inventions encompassed within this specification may also include embodiments that are only partially mentioned or alluded to or are not mentioned or alluded to at all in this brief summary or in the abstract.

### BRIEF DESCRIPTION OF THE FIGURES

Although the following figures depict various examples of the invention, the invention is not limited to the examples depicted in the figures.

FIGS. 1A and B show diagrams of an embodiment of the multiplex digital PCR for two non-polymorphic targets. FIG. 1A shows an example of the results when two target loci are unlinked. FIG. 1B shows an example of the results when two target loci are linked on a continuous strand of DNA.

FIG. 2 shows a diagram of an embodiment of the multiplex digital PCR for multiple polymorphic targets.

FIG. 3 shows data obtained for a multiplex PCR for multiple targets using methods of the invention.

FIGS. 4A and B show a method to identify splice isoforms in lung RNA using embodiments of the methods described herein. FIG. 4A shows a map of the targets. FIG. 4B shows an example of the results using multiplex preamplification.

FIGS. 5A and B show a method to detect and quantify mRNA isoforms using multiplex digital PCR. FIG. 5A shows a map of the targets. FIG. 5B shows an example of the results.

### DETAILED DESCRIPTION

Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

In general, the specification provides methods and compositions for identifying the presence of linkage between two or more targets in a sample using multiplex digital PCR. The targets can be any nucleic acids that are not separated by dilution of the sample (e.g., have a physical linkage).

The methods and examples herein provide methods and examples demonstrating detection of physical linkages in both DNA and RNA target sequences using digital PCR with or without preamplification.

Definitions and General Methods:

The practice of the present invention will employ, unless otherwise indicated, conventional methods of chemistry, biochemistry, molecular biology, virology, immunology and pharmacology, within the skill of the art. Such techniques are explained fully in the literature.

In the description that follows, a number of terms used in chemistry, biochemistry, molecular biology, virology, immunology and pharmacology are extensively utilized. In order to provide a clearer and consistent understanding of the specification and claims, including the scope to be given such terms, the following definitions are provided.

As used in this specification and the appended claims, the singular forms “a,” “an” and “the” include plural references unless the content clearly dictates otherwise.

As used herein “nucleotide” refers to a base-sugar-phosphate combination. Nucleotides are monomeric units of a nucleic acid sequence (DNA and RNA) and deoxyribonucleotides are “incorporated” into DNA by DNA polymerases. The term nucleotide includes deoxyribonucleoside triphosphates such as dATP, dCTP, dITP, dUTP, dGTP, dTTP, or derivatives thereof. Such derivatives include, for example, [aS]dATP, 7-deaza-dGTP and 7-deaza-dATP. The term nucleotide as used herein also refers to dideoxyribonucleoside triphosphates (ddNTPs) and their derivatives. Illustrative examples of dideoxyribonucleoside triphosphates (ddNTPs) include, but are not limited to, ddATP, ddCTP, ddGTP, ddITP, and ddTTP.

The term “nucleic acid or nucleotide analogs” refers to analogs of nucleic acids made from monomeric nucleotide analog units, and possessing some of the qualities and properties associated with nucleic acids. Nucleotide analogs may have modified (i) nucleobase moieties, e.g., C-5-propyne pyrimidine, pseudo-isocytidine and isoguanosine, (ii) sugar moieties, e.g., 2′-O-alkyl ribonucleotides, and/or (iii) internucleotide moieties, e.g., 3′-N-phosphoramidate. See Englisch, U. and Gauss, D. “Chemically modified oligonucleotides as probes and inhibitors”, Angew. Chem. Int. Ed. Engl. 30:613-29 (1991). A class of analogs where the sugar and internucleotide moieties have been replaced with an 2-aminoethylglycine amide backbone polymer is peptide nucleic acids PNA. See P. Nielsen et al., Science 254:1497-1500 (1991).

As used herein the terms “hybridization” and “hybridizing” refer to the pairing of two complementary single stranded nucleic acid molecules (RNA and/or DNA) to give a double stranded molecule. As used herein, two nucleic acid molecules may be hybridized, even if the base pairing is not completely complementary. Accordingly, mismatched bases do not prevent hybridization of two nucleic acid molecules provided that appropriate conditions, well known in the art, are used.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other.

As used herein “primer” refers to a single stranded oligonucleotide that is extended by covalent bonding of nucleotide monomers during amplification or polymerization of a template molecule.

As used herein “probe” refers to a single stranded oligonucleotide that hybridizes to a complementary nucleic acid.

The terms “nucleic acid,” “polynucleotide,” “oligonucleotide” or “oligo” mean polymers of nucleotide monomers or analogs thereof, including double and single stranded deoxyribonucleotides, ribonucleotides, alpha-anomeric forms thereof, and the like. Usually, the monomers are linked by phosphodiester linkages, where the term “phosphodiester linkage” refers to phosphodiester bonds or bonds including phosphate or analogs thereof, including associated counterions, e.g., H⁺, NH₄⁺, Na⁺.

The term “nucleotide triphosphate or analog thereof” refers to combinations of one or more ribonucleotide triphosphates (GTP, UTP, CTP and ATP or nucleotide analogs thereof), or deoxyribonucleotide triphosphates dATP (dGTP, dTTP, dCTP or deoxyribonucleotide analogs thereof); adenosine 5′-phosphosulfate (APS); and D-luciferin and/or analogs of either.

As used herein “fluorophore,” “fluorescent moiety,” “fluorescent label” and “fluorescent molecule” refer to a molecule, label or moiety that has the ability to absorb energy from light, transfer this energy internally, and emit this energy as light of a characteristic wavelength.

As used herein “dyes” are any type of moiety that can make the positive reactions distinguishable. Examples of dyes include fluorophores, common dyes, intercolators, quantum dots and radioactive moieties. Other dyes are discussed in the section entitled “dyes” herein.

“Target molecule” or “target” or “target locus”, as used herein, refers to a nucleic acid molecule to which a particular primer or probe is capable of preferentially hybridizing.

“Target sequence”, as used herein, refers to a nucleic acid sequence within the target molecules to which a particular primer is capable of preferentially hybridizing.

As used herein “SNP” means a single nucleotide polymorphism. Single nucleotides may be changed (substitution), removed (deletions) or added (insertion) to a polynucleotide sequence. Insertion or deletion SNPs (InDels) may shift translational frame.

As used herein “mutation” refers to any type of change in nucleotide sequence. Mutations can include multiple nucleotide polymorphisms, deletions and/or insertions.

As used herein “translocation” or “chromosome translocation” refers to a chromosome abnormality caused by rearrangement of parts between nonhomologous chromosomes. A gene fusion may be created when the translocation joins two otherwise separated genes, the occurrence of which is common in cancer. It can be detected on cytogenetics or a karyotype of affected cells. Translocations can be balanced (in an even exchange of material with no genetic information extra or missing, and ideally full functionality) or unbalanced (where the exchange of chromosome material is unequal resulting in extra or missing genes).

As used herein “linkage” refers to any type of physical linkage between two targets. In some embodiments the linkage allows that the two targets do not come apart when diluted.

As used herein “limiting dilution” refers to a dilution of a nucleic acid until there is less than 1 per designated volume. As used herein, limiting dilution refers to the number of targets and/or nucleic acid molecules in a well. The sample is diluted to a point in which less than 1 copy will be present in a well. Alternatively, the sample can be diluted to a point in which there is one copy/4 wells or other set ratio of copies/well. Other possibilities are discussed herein with reference to the methods.

As used herein “random distribution” refers to the non-deterministic physical distribution of nucleic acids or other targets into individual reactions in a set of reactions.

As used herein a “student's T test” refers to a statistical hypothesis test in which the test statistic follows a Student's t distribution if the null hypothesis is supported. It is most commonly applied when the test statistic would follow a normal distribution if the value of a scaling term in the test statistic were known. When the scaling term is unknown and is replaced by an estimate based on the data, the test statistic (under certain conditions) follows a Student's t distribution. As used herein the “p-value” is the probability of obtaining a test statistic at least as extreme as the one that was actually observed, assuming that the null hypothesis is true. The lower the p-value, the less likely the result is if the null hypothesis is true, and consequently the more “significant” the result is, in the sense of statistical significance. One often accepts the alternative hypothesis, (i.e. rejects a null hypothesis) if the p-value is less than 0.05 or 0.01. 0.05 or 0.01 corresponds to a 5% or 1% chance respectively of rejecting the null hypothesis when it is true.

As used herein “F-test” refers to any statistical test in which the test statistic has an F-distribution (a continuous probability distribution) under the null hypothesis.

As used herein “Chi-square test” refers to any statistical test in which the test statistic has a Chi-square distribution. A Chi-square distribution in probability theory and statistics, the chi-square distribution (also chi-squared or distribution) with k degrees of freedom is the distribution of a sum of the squares of k independent standard normal random variables.

As used herein “Fisher-test” refers to a statistical significance test used in the analysis of contingency tables where sample sizes are small. It is one of a class of exact tests, so called because the significance of the deviation from a null hypothesis can be calculated exactly, rather than relying on an approximation that becomes exact in the limit as the sample size grows to infinity, as with many statistical tests.

As used herein “ANOVA test” refers to an analysis of variance test and is a collection of statistical models, and their associated procedures, in which the observed variance in a particular variable is partitioned into components attributable to different sources of variation. In its simplest form ANOVA provides a statistical test of whether or not the means of several groups are all equal, and therefore generalizes t-test to more than two groups.

As used herein “multiple-comparison ANOVA test” refers to a type of analysis of variance test in which one considers a set, or family, of statistical inferences simultaneously.

As used herein “preamplification” (or “PreAmplification” or “PreAmp”) is a method to amplify a small sample to allow for a greater number of reiterations. For example, if there is not enough DNA in a single cell to do a large number of multiplex reactions, the sample from the single cell can be amplified and then aliquoted out to perform a number of different reactions on that one sample. See, for example, U.S. Pat. Nos. 6,605,451 and 7,087,414, both of which are hereby incorporated by reference in their entireties. As used herein, the mother well is preamplified to allow aliquoting into a daughter well.

As used herein “polymorphic” refers to the simultaneous occurrence in the same locus of two different alleles. For example, one type of allele might be found on the maternal chromosome and a different allele on the paternal chromosome.

As used herein “wells” refers to any container or surface that can contain a target nucleic acid sample and keep it from mixing with a second sample. Exemplary “wells” include wells in a micro-well plate, tubes (e.g., microfuge tubes), capillaries, emulsions, arrays of miniaturized chambers, and nucleic acid binding surfaces. A “mother well” refers to a first well from which a sample can be aliquoted to other wells, called daughter wells. A “daughter well” refers to one or more wells that a sample from a mother well can be aliquoted into.

General Description

In general, the specification provides methods and compositions for identifying the presence of linkage between two or more targets in a sample using multiplex digital PCR. The targets can be any nucleic acids that are not separated by dilution of the sample (e.g., have a physical linkage). For example, if two targets are on the same chromosome, they will be linked. Alternatively, if an mRNA is undergoing translation, it will be linked to an rRNA. This is another type of linkage. The methods also have the advantage of identifying the linkage at high resolution. The methods also have the advantage of quantitating the linkage. For example, the breakpoint for a chromosomal translocation can be localized at a higher resolution than a method such as FISH is capable of localizing.

Linkage can be used to localize areas of disease risk across the genome and/or to identify targets associated with a specific disease. These targets can be used to identify, diagnose and/or prognose a disease in a sample.

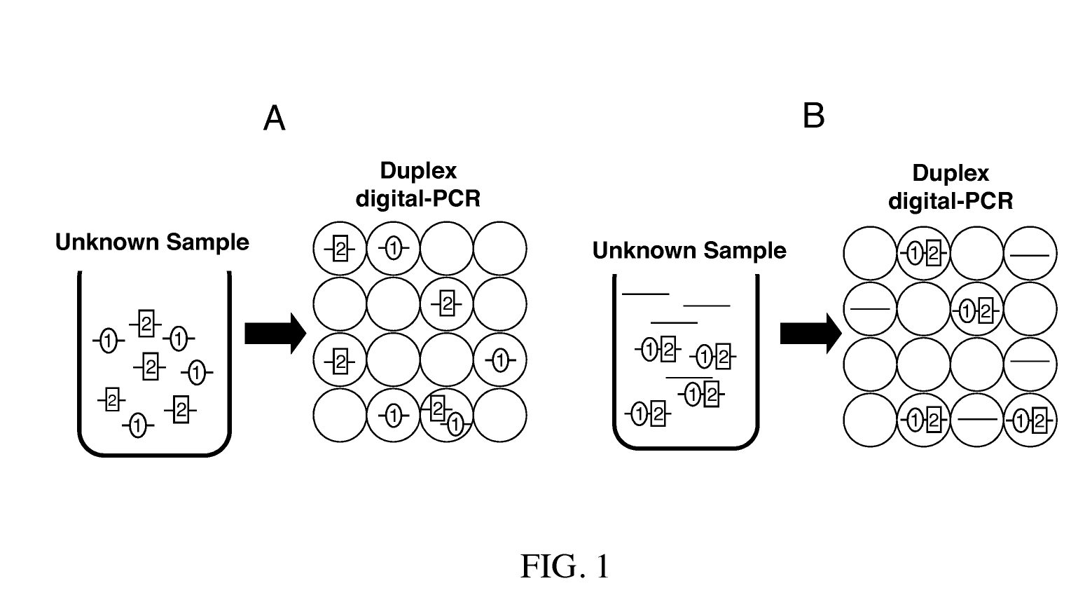

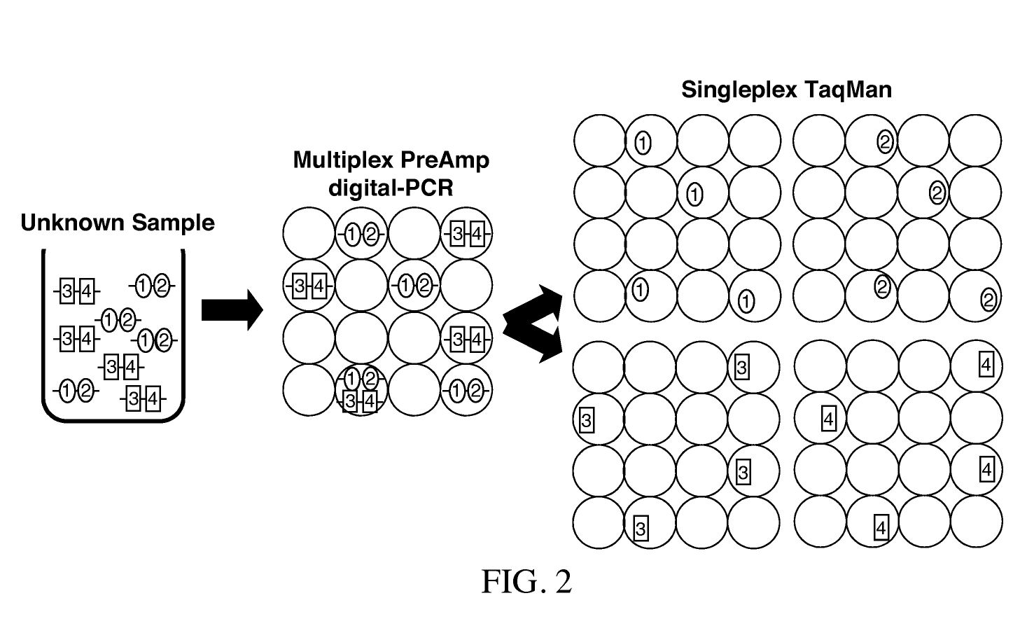

Multiplex digital PCR is a method which exploits the disclosed system to determine whether two or more target DNA and/or RNA sequences are physically linked. Specifically, when a pool of template is randomly distributed among multiple PCR reactions, physical linkage of two or more sequences in that pool will cause them to act as a single unit. This in turn will result in a highly non-random distribution relative to each other (see FIG. 1). The distribution of multiple target sequences can be detected by performing the initial PCR reaction in multiplex, either through the use of multiple dyes or multiplex PreAmplification (see FIG. 2).

The methods and examples herein provide methods and examples demonstrating detection of physical linkages in both DNA and RNA target sequences using digital PCR with or without preamplification.

Methods

In general, the specification provides methods and compositions for identifying the presence of linkage between two targets in a sample using multiplex digital PCR. The methods can be one-step methods or two-step methods. In two-step methods the first and second PCR can use the same primers or different primers, including, but not limited to nested primers, chimeric primers, or universal primers.

One-Step Methods

In some embodiments, one-step methods do not involve preamplification. The amount of multiplexing in one-step methods is constrained by the number of different dyes which are available. For example, there are six commercially available dyes that are believed to be known at this time. However, improvements in dye spectral overlap, quantum dyes, the spectral resolution and differentiation capability of instruments, spectral analysis algorithms, or combinatorial analysis methods could possibly increase the number of dyes which can be utilized in one-step methods. This method can be limited by the size of the PCR. Thus, for example, the method can be used for targets of between about 1 and 100 base pairs, including but not limited to: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99 targets. It is possible that, depending on the dyes that become available, the number of targets could increase to more than 100.

With reference to FIG. 1, for two non-polymorphic targets, Target 1 is shown as an oval with the number “1” in it and Target 2 is shown as a rectangle with the number “2” in it. TaqMan® assays (e.g., using FAM and VIC probes) can be run in duplex qPCR reactions. PreAmplification is not usually required unless the amount of nucleic acid in the sample is very low. The sample can be diluted to a limiting dilution. A statistical analysis to determine whether co-detection of the two targets (“1” and “2”) is random (not linked=null hypothesis) or non-random (physically linked) can be used to determine the original linkage state. In FIG. 1A, the unknown sample is shown as a mixture of Targets “1” and “2”. After Duplex digital-PCR, the positive results are shown in the respective wells. For example, where two target loci are unlinked, a random distribution of DNA strands into multiple assay reactions will distribute them randomly relative to each other. Statistical analysis of example data (1/16 positive for target 1 and 2, 3/16 positive for target 1 only, 3/16 positive for target 2 only, 9/16 positive for neither) gives a Chi-square equal to 0 and a P-value equal to 1.0. Thus, in this case, there is no evidence of physical linkage between target 1 and 2. In FIG. 1B, where target loci are linked on a continuous strand of DNA, a random distribution of DNA strands into multiple assay reactions will distribute the targets into the same wells. Statistical analysis of the example data shown (4/16 positive for target 1 and 2, 0/16 positive for target 1 only, 0/16 positive for target 2 only, 12/16 positive for neither) gives a Chi-square equal to 16 and a P-value equal to 0.001. Thus, there is strong evidence of physical linkage between target 1 and 2.

In some embodiments, the methods include the steps of diluting the sample via limiting dilution; aliquoting the diluted sample into wells; performing multiplex PCR in each chamber, wherein a first dye is used for the first target and a second dye is used for the second target; and identifying the presence of the first and second dyes in the wells, wherein a non-random distribution of the dyes identifies the targets as linked.

In some embodiments, each of the steps of the one-step method is a distinct step. In another embodiment, although discussed as distinct steps, they may not be distinct and/or separate steps. In other embodiments, the one-step method may not have all of the above steps and/or may have other steps in addition to or instead of those listed above. The steps of the one-step method may be performed in another order. Subsets of the steps listed above as part of the one-step method may be used to form their own method.

Two-Step Methods

The two step methods (also called PreAmp digital PCR) involve an initial preamplification step which allows for an increased multiplicity. The first step involves amplifying the target using specific primers in a mother reaction. Then the product of the reaction can be aliquoted and daughter reactions performed to identify whether the targets were present in the original mother reaction. The identification can be by singleplex PCR, by multiplex PCR, and/or by hybridization methods (in a well, in a micro-well plate, capillaries, emulsions, arrays of miniaturized chambers, and nucleic acid binding surfaces).

In some embodiments, these methods are not limited by the size of the PCR as long as the targets are physically linked.

In some embodiments, the same primers are used for both the mother and daughter reactions. In some embodiments, different primers are used, such as nested, chimeric and/or universal primers. In some embodiments, the targets are identified as linked by a non-random distribution wherein the non-random distribution has a p-value of <0.05 (e.g., less than 5% of the time you expect to see a particular distribution). In some embodiments, the targets are identified using a target-specific probe. In some embodiments, the targets are identified using primers that contain a dye.

With reference to FIG. 2, for multiple and/or polymorphic targets, multiplex TaqMan® PreAmp is used for the initial digital-PCR reaction. The Figure shows the presence of four targets in the Unknown Sample, numbered “1”, “2”, “3”, and “4”. Targets “1” and “2” are designated as ovals. Targets “3” and “4” are designated as rectangles. The Unknown Sample is used in Multiplex PreAmp digital PCR reactions and primers for all of the targets being tested are present in the PreAmp reaction. The PreAmp product from each well can be used as a template for singleplex reactions on each target to determine which targets were present in the original reactions. Thus, the mother reactions can be preamplified. In some embodiments, the result of the preamplification in the mother wells are then diluted into, for example, 16 wells and each target is assayed as a singleplex TaqMan® reaction. These are the daughter reactions. The results in the daughter reactions in FIG. 2 show that targets “1” and “2” are linked and targets “3” and “4” are linked.

In some embodiments, the methods include the steps of diluting the sample into mother wells to a limiting dilution; preamplifying the diluted sample in the wells using multiplex PCR without a dye; aliquoting the product of each mother well into multiple daughter wells; determining the presence of a first target using PCR with a dye; determining the presence of a second target using PCR with a dye; and identifying whether the first and second target are linked by identifying mother wells which generated a nonrandom distribution of the first target and second target in its daughter wells. In some embodiments, the PCR in the daughter wells uses primers having the same sequence as the primers used in the mother wells. In some embodiments, the PCR in the daughter wells is via nested PCR. In some embodiments, the dye is attached to a probe. In some embodiments, the dye is attached to a primer.

In some embodiments, each of the steps of the two-step method is a distinct step. In another embodiment, although discussed as distinct steps, they may not be distinct and/or separate steps. In other embodiments, the two-step method may not have all of the above steps and/or may have other steps in addition to or instead of those listed above. The steps of the two-step method may be performed in another order. Subsets of the steps listed above as part of the two-step method may be used to form their own method.

Limiting Dilution

Digital PCR involves diluting a sample to a limiting dilution. As used herein, limiting dilution refers to the number of targets and/or nucleic acid molecules in a well. The sample is diluted to a point in which less than 1 copy will be present in a well. While the amount of dilution may depend on a number of factors, including the numbers of copies of a target in a genome, the quality of the DNA or RNA being used (e.g., whether it is partially degraded), the preparation of the nucleic acid sample, and the amount of the nucleic acid sample, at some point the sample can be diluted to a limiting dilution. Various aspects can be taken into account when deciding how much the sample needs to be diluted to obtain a limiting dilution, including but not limited to, number of chromosomes, number of targets, number of copies of the target, and amount of degradation.

If the number of targets and the concentration is known, the dilution scheme can be identified without first doing a test PCR. If not, a test PCR can be performed. In some embodiments, the sample is diluted to a limiting dilution before amplification and detection (e.g., the one-step procedure described herein). In some embodiments, the sample is preamplified and then diluted to a limiting dilution to amplify and detect the target (e.g., the two-step procedure described herein).

In some embodiments, limiting dilution is from about 1 target per well to about 1 target per 10 wells, including 1 target/2 wells, 1 target/3 wells, 1 target/4 wells, 1 target/5 wells, 1 target/6 wells, 1 target/7 wells, 1 target/8 wells, and 1 target/9 wells.

Digital PCR

The methods and compositions discussed herein use digital PCR to identify linkage. The type of digital PCR can be any type known to the skilled artisan. In some embodiments, the digital PCR is also multiplexed. In some embodiments, the digital PCR includes the use of a probe. In some embodiments, the digital PCR does not include the use of a probe. It is understood that any type of PCR can be performed in a digital manner. For example, the target nucleic acid can be diluted such that there is one or less copies of the target per well and the PCR performed.

PCR methods useful in accordance with the present methods and compositions include, but are not limited to, FRET, TaqMan®, Molecular Beacons, Amplifluor®, Scorpion™ Plexor™, or BHQplus™.

Targets

Any nucleic acid target can be used without limitation. In some embodiments, the target can be identified using PCR. In some embodiments, the two or more targets are the same type of nucleic acid. In some embodiments, the two or more targets are different types of nucleic acid (e.g., an mRNA and chromosomal DNA). In some embodiments, the targets can be chosen as anything that might be in physical linkage. The targets can be DNA, RNA (mRNA, rRNA, tRNA, mitochondrial RNA), or any type of nucleic acid. The targets can be genes, loci, SNPs, microsattelites, translocations, alleles, mutations (e.g., multiple nucleotide and/or large deletions, additions, or changes), multi-RNA complexes, splice variants, transposons, ribozymes, microRNAs (primary, pre- or mature microRNAs), bacterial genomes, plasmids, viral genomes, and viroids. For example, in some embodiments, DNA is used to identify the presence of a SNP. In some embodiments, RNA is used to identify the expression of a SNP. The targets can be SNPs, InDels, mutations, translocations, inversions, duplications, deletions, ring chromosomes, isochromosomes, splice regions, microsatellites, mature microRNAs, pri-microRNAs, pre-microRNAs, non-coding RNAs, mRNAs primary transcripts, genomic loci, alleles, multi-RNA complexes, splice variants, transposons, ribozymes, bacterial genes, viral nucleic acids, ribosomal RNAs, viral insertion sites, vector insertion sites, hypervariable regions, mitochondrial DNA, highly polymorphic regions, MHC regions, and MHC gene products. In some embodiments, the target is a splice junction to identify mRNA isoforms.

In some embodiments, the target is polymorphic. For example, one copy is on the maternal chromosome and one copy is on the paternal chromosome.

The number of targets is not constrained in any way. For example, if there are more than about 384 targets, the one-step method may be difficult. Thus, for the one-step method, the number of targets can be between about 2 and about 384, including but not limited to, between about 2 and about 300, between about 2 and about 200, between about 2 and about 100, between about 2 and about 75, between about 2 and about 50, between about 2 and about 40, between about 2 and about 30, between about 2 and about 25, between about 2 and about 20, and between about 2 and about 10.

However, the 2-step method can be used and may increase the number of possible targets to be assayed in a single assay up to about 10,000. Thus, a preamplification can be performed on the initial sample and then aliquoted into up to about 10,000 separate reactions to be amplified and detected. In some embodiments, the number of reactions is between about 2 and about 100,000, including between about 2 and about 10,000, including between about 2 and about 1000, including between 2 and about 100.

Dyes

The dyes can be any dye that allows for the identification of the locus. Dyes can be attached to a primer or probe. Alternatively, dyes can be used that intercolate into double stranded DNA (e.g., SYBR). Dyes can include simple dyes (e.g., pico green), radioactive moieties, fluorescent dyes, and quantum dots, for example.

A dye can be attached to a primer or a probe (an oligonucleotide). The dye can be easily incorporated internally to an oligonucleotide and/or can be incorporated at the 5′ end of an oligonucleotide primer or probe. The dye can be located on the primer or a probe. In some embodiments, the dye is located on the primer internally, but not at the 3′ end if it inhibits primer extension by an enzyme.

In some embodiments, the dye is a fluorophore and the fluorophore is a commonly used fluorophore. Fluorophores that are commonly used in FRET include fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-1-sulfonic acid (EDANS). The Fluorophore can be any fluorophore known in the art, including, but not limited to: FAM, TET, HEX, Cy3, TMR, ROX, Texas red, LC red 640, Cy5, and LC red 705.

Other fluorophores can be chosen from: 4-acetamido-4′-isothiocyanatostilbene-2,2′disulfonic acid; acridine and derivatives (e.g., acridine, acridine isothiocyanate); 5-(2′-aminoethyl)aminonaphthalene1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives (e.g., coumarin, 7-amino-4-methylcoumarin, 7-amino-4-trifluoromethylcoumarin); cyanosine; 4′,6-diaminoidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetraimine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives (e.g., eosin, eosin isothiocyanate); erythrosine and derivatives (e.g., erythrosine B, erythrosine isothiocyanate); ethidium; fluorescein and derivatives (e.g., 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate, and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives (e.g., pyrene, pyrene butyrate, succinimidyl 1-pyrene butyrate); Reactive Red 4 (Cibacron Brilliant Red 3B-A); rhodamine and derivatives (e.g., 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine (tetramethyl rhodamine isothiocyanate (TRITC)); riboflavin; rosolic acid; and terbium chelate derivatives.

Fluorophores for use in the present invention may be obtained commercially, for example, from Biosearch Technologies (Novato, Calif.), Life Technologies (Carlsbad, Calif.), GE Healthcare (Piscataway N.J.), Integrated DNA Technologies (Coralville, Iowa) and Roche Applied Science (Indianapolis, Ind.). In some embodiments, the fluorophore is chosen to be usable with a specific detector, such as a specific spectrophotometric thermal cycler, depending on the light source of the instrument. In some embodiments, if the assay is designed for the detection of two or more targets (e.g., multiplex assays), two or more different fluorophores can be chosen with absorption and emission wavelengths that are well separated from each other (e.g., having minimal spectral overlap).

Exemplary radioactive moieties include Phosporus 32, Tritium, technetium-99m, Iodine 125 or any other moieties known in the art.

Intercolative dyes are dyes that bind to double-stranded nucleic acids (e.g., DNA) and sometimes single-stranded nucleic acids. Exemplary intercolative dyes include, but are not limited to, SYBR Green I, SYBR Green II, SYBR Gold, YO (Oxazole Yellow), TO (Thiazole Orange), PG (PicoGreen).

A quantum dot is a semiconductor whose excitons are confined in all three spatial dimensions. Consequently, such materials have electronic properties intermediate between those of bulk semiconductors and those of discrete molecules. Stated simply, quantum dots are semiconductors whose electronic characteristics are closely related to the size and shape of the individual crystal. Generally, the smaller the size of the crystal, the larger the band gap, the greater the difference in energy between the highest valence band and the lowest conduction band becomes, therefore more energy is needed to excite the dot, and concurrently, more energy is released when the crystal returns to its resting state. For example, in fluorescent dye applications, this equates to higher frequencies of light emitted after excitation of the dot as the crystal size grows smaller, resulting in a color shift from red to blue in the light emitted. In addition to such tuning, a main advantage with quantum dots is that, because of the high level of control possible over the size of the crystals produced, it is possible to have very precise control over the conductive properties of the material.

In some embodiments, the dye is chosen from: FAM, TET, HEX, Cy3, TMR, ROX, Texas red, LC red 640, Cy5, VIC, SYBR, LC red 705, JUN, ABY, TED, TZA, and SID.

Linkage and Methods of Quantifying Linkage

The disclosed methods and compositions are, in part, derived from the identification that if digital PCR is used, the distribution of targets is random. So, by multiplexing the linkage status, the linkage of two or more targets can be identified by analyzing whether there is a random or nonrandom distribution. If the PCR results show a nonrandom distribution of targets, those targets can be considered linked. In standard digital PCR, a large number of identical PCRs targeting a single amplicon are typically run on a single sample that is dilute enough for most reactions to randomly contain either zero or one target molecule (e.g., limiting dilution). If the number of reactions represents a large enough statistical sample, the absolute number of target molecules in the original sample can be calculated from the frequency of wells in which amplification occurs. Since the distribution of target templates is random, the probability of observing amplification in any given reaction in a set of reactions is purely random relative to the other reactions in the set.

Any type of physical linkage can be detected without exception as long as the linkage of the targets is not affected by dilution of the sample (e.g., the targets do not separate). Examples include, but are not limited to, two targets on a single chromosome, two targets on a single bacterial genome, a microRNA and its target, nascent primary RNA attached to genomic DNA, ribosomal RNA and RNAs that they are attached to.

The methods can be used to determine the linkage between one or more alleles of a polymorphic site, to determine haplotype or haplogroup, for DNA profiling, DNA testing, DNA typing genetic fingerprinting, ancestry analysis, paternity testing, or maternity testing, for disease association, disease risk assessment, patient stratification, drug metabolism analysis or diagnosis.

The non-random distribution can be determined using any methods known in the art, including but not limited to, Student's T-test, F-test, Chi-square test, Fisher-test, ANOVA test, or multiple comparison ANOVA test.

Diseases and Samples

In some embodiments, the methods can be used to identify linkage between two or more targets. The methods can also be used to more clearly identify the breakpoint for a known or unknown translocation. The methods can be used in the detection, diagnosis, and prognosis of disease. Thus, targets can be chosen that can detect, diagnose and/or prognose the stage of a disease. The methods can be used to identify whether a target is involved in the detection, diagnosis and/or prognosis of a disease. The methods can be used to identify whether a disease will respond to treatment (e.g., whether a cancer will respond to a particular chemotherapy treatment).

Translocations can be definitive for a particular disease. Some human diseases that involve translocations include, but are not limited to: (1) Cancer—several forms of cancer are caused by translocations. This has been described mainly in leukemia (acute myelogenous leukemia and chronic myelogenous leukemia). (2) Infertility—this can occur when one of the would-be parents carries a balanced translocation, where the parent is asymptomatic but conceived fetuses are not viable. For example, Down's syndrome is caused in a minority (5% or less) of cases by a Robertsonian translocation of about a third of chromosome 21 onto chromosome 14.

Exemplary diseases involving linkage include, but are not limited to cancers and leukemias, genetic diseases, neurological diseases (e.g., Alzheimer's disease, moyomoya disease, Parkinson's disease), digestive diseases (e.g., celiac disease), heart disease, diabetes, autoimmune diseases, infectious disease (e.g., susceptibility), and CNS disease (e.g., Pelizaeus-Merzbacher disease).

Chromosomal abnormalities, in particular translocations and their corresponding gene fusions, have an important role in the initial steps of tumorigenesis. Acquired chromosome changes have now been reported in more than 50,000 cases across all main cancer types. An analysis of available data shows that gene fusions, in particular, occur in all malignancies. Currently, 358 gene fusions involving 337 different genes have been identified. An increasing number of gene fusions are being recognized as important diagnostic and prognostic parameters in malignant hematological disorders and childhood sarcomas and some may be definitive or causative of the cancer.

The methods described herein can be used to identify chromosome translocations. Chromosome translocations are typically described according to an internationally accepted nomenclature. For example, regions and bands along each chromosome arm are numbered consecutively from the centromere outward. The symbols p and q are traditionally used to designate the short and long arms of each chromosome, respectively. In designating a particular band, four items are required; the chromosome number, the arm symbol, the region number and the band number within that region. For example, 9q34 indicates chromosome 9, long arm, region 3, band 4. Letter designations are used to specify the type of rearrangements, for example, “t” for translocation, “inv” for inversion, and “ins” for insertion. In the description of an abnormality, this designation is followed by the structurally altered chromosomes, separated by a semicolon, within parentheses; the breakpoints are then specified within another parenthesis and are listed in the same order as the chromosomes involved, again separated by a semicolon. Thus, t(9;22)(q34;q11) signifies a translocation between chromosomes 9 and 22 with breakpoints in bands 9q34 and 22q11 respectively.

The first specific translocation identified in human neoplasia was t(9;22)(q34;q11), resulting in the Philadelphia chromosome. Another example is Burkitt lymphoma which harbors one of three translocations t(8;14) (q24;q32), t(2;8)(p11;q24), and t(8;22)(q24;q11) and in all three cases, the breakpoint in chromosome 8 is within or adjacent to the MYC gene. Although there is usually a very good association between specific tumor types and specific chimeric genes, there are some notable exceptions. The most striking is t(12;15)(p13;q25), leading to the fusion of ETV6 with the neurotrophic tyrosine kinase receptor type 3 gene (ETV6-NTRK3), which occurs in tumors of totally different histogenetic derivations, namely AML, mesoblastic nephroma of the kidney, soft tissue fibrosarcoma and adenocarcinoma of the breast. Other examples of translocations and their clinical ramifications include, but are not limited to, t(1;22)(p13;q13)—Acute megakaryoblastic leukemia; t(2;5)(p23;q35)—Anaplastic large T-cell lymphoma; t(8;14)(q24;q32)—Burkitt lymphoma/leukemia (highly aggressive but with a good prognosis); t(8;21)(q22;q22)—Acute myeloid leukemia; t(9;22)(q34;q11)—Chronic myeloid leukemia; t(12;21)(p13q22)—B-cell precursor acute lymphoblastic leukemia; t(14;16)(q32;q23)—Multiple myeloma; t(15;17)(q22;q21)—Acute promyelocytic leukemia; t(X;1)(p11;q23)—Papillary renal cell carcinoma; t(2;3)(q13;p25)—Follicular thyroid carcinoma; t(7;16)(q33;p11)—low-grade malignant fibromyxoid soft tissue sarcoma; t(7;17)(p15;q11)—Endometrial stromal sarcoma; t(9;22)(q32;q12)—Soft tissue chondrosarcoma with abundant myxoid matrix; t(11;22)(p13;q12)—Desmoplastic small round cell tumor; t(11;22)(q24;q12)—Ewing sarcoma; t(15;19)(q14;p13)—Poorly differentiated carcinoma affecting midline structures in children and adolescents.

Some translocations, while not always associated with a cancer, can be periodically associated with the cancer and can affect aggressiveness, prognosis, and/or the ability to treat the cancer with specific chemotherapeutic agents. For example, a small proportion of patients with chronic myeloproliferative diseases have constitutive activation of the gene for platelet-derived growth factor receptor beta (PDGFRβ), which encodes a receptor tyrosine kinase. The gene is located on chromosome 5q33, and the activation is usually caused by a t(5;12)(q33;p13) translocation associated with an ETV6-PDGFR/3 fusion gene. The tyrosine kinase inhibitor imatinib mesylate specifically inhibits ABL, PDGFR, and KIT kinases and has impressive clinical efficacy in BCR-ABL—positive chronic myeloid leukemia.

Thus, it is of interest to identify chromosomal abberrations in cancers, whether they are specifically prognostic of the cancer, a type of cancer, the ability to treat the cancer effectively or the aggressiveness of the cancer.

Detection

The detection of the signal can be made using any reagents and/or instruments that detect the dye being used (e.g., a simple dye, a radioactive moiety and/or a fluorescent dye). For example, the reagent and/or instrument may detect a change in fluorescence from a fluorophore. Fluorescent measurements can be made using a fluorometer, plate reader with fluorescent detector or a real-time PCR thermocycler. Examples of spectrophotometric thermal cyclers include, but are not limited to, Applied Biosystems (AB) PRISM® 7000, AB 7300 real-time PCR system, AB 7500 real-time PCR system, AB PRISM® 7900HT, Bio-Rad ICycler IQ™, Cephied SmartCycler® II, Corbett Research Rotor-Gene 3000, Idaho Technologies R.A.P.I.D.™, MJ Research Chromo 4™, Roche Applied Science LightCycler®, Roche Applied Science LightCycler®2.0, Stratagene Mx3000P™, and Stratagene Mx4000™. It should be noted that new instruments are being developed at a rapid rate and any instruments could be used for the methods.

Radioactivity can be detected using any methods known to the skilled artisan, including but not limited to a scintillation counter, film, silver halide emulsion, a Geiger counter, Phosphor storage screen, micro-autoradiography imager, or electron microscopy.

Simple dyes can be detected visually. However, they may be quantitated using any method known to the skilled artisan, including but not limited to spectroscopy, film, or electronic image sensor.

Polymorphic and Non-Polymorphic Loci

The methods and compositions can be used to identify the linkage of polymorphic and non-polymorphic loci as the targets. In this case, polymorphic can mean that there may be two copies of a locus (e.g., gene). For example, there might be a maternal copy and a paternal copy and the two genes could be different. For example, one copy could carry a mutation, SNP, translocation or other difference. When a translocation is identified, typically, one chromosome will show linkage of the two translocated targets and the other will not (e.g., the maternal and paternal chromosomes).

Kits

Some embodiments of the invention provide kits for the identification of linkage between two or more targets using multiplex digital PCR. The kits may include at least one of a primer and/or a probe that binds specifically to one or more targets to identify linkage. The kits may also include reagents for preamplification, multiplex amplification, and/or singleplex amplification. Some embodiments of the invention provide kits for the identification of linkage between two or more targets in a sample, including at least one primer and/or probe for each target. The kit can include primers and/or a probe for any of up to 10,000 different targets to be identified in a single assay. The primers and/or probe can include a different dye for each of the targets to be identified. The kit can be used in any of the methods known or disclosed herein. Some embodiments of the kit are used for identifying the linkage between two targets on a single chromosome, two targets on a single bacterial genome, a microRNA and its target, nascent primary RNA attached to genomic DNA, ribosomal RNA and/or RNAs that they are attached to.

The kits may contain reagents to determine the linkage between one or more alleles of a polymorphic site, to determine haplotype or haplogroup, for DNA profiling, DNA testing, DNA typing, genetic fingerprinting, ancestry analysis, paternity testing, or maternity testing, for disease association, disease risk assessment, patient stratification, drug metabolism analysis or diagnosis using methods taught herein.

Any embodiments discussed with respect to compositions and/or methods of the invention, as well as any embodiments in the Examples, is specifically contemplated as being part of a kit.

The components of the kits may be packaged either in aqueous media, in anhydrous buffers or in lyophilized form. The container means of the kits will generally include at least one vial, test tube, flask, bottle, syringe or other container means, into which a component may be placed, and preferably, suitably aliquoted. Where there is more than one component in the kit (e.g., especially when packaged together), the kit also will generally contain a second, third or other additional container into which the additional components may be separately placed. However, various combinations of components may be comprised in a vial. The kits can also include a means for containing the reagents, and any other reagent containers in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred.

However, the components of the kit may also be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

A kit can also include instructions for employing the kit components as well as the use of any other reagent not included in the kit. Instructions can include variations that can be implemented.

Samples

The methods and compositions can be used for detection of linkage between one or more targets in a sample. The sample can include one or more targets. The sample can be purified or unpurified. The sample can be a biological sample that has been treated to be used in the methods of the invention. In some embodiments, if the biological sample does not interfere with the methods of the invention, it can be used untreated (and/or unpurified). The sample can be purified nucleic acid from a biological source. Exemplary biological material includes cells, tissues, blood products, bodily fluids, and viral material. The biological material can be from a patient, for example, from a human or animal. The methods can also be used with nucleic acids from a sample that could be damaged. For example, one valuable use for the method is FFPE (formalin-fixed paraffin embedded tissue). This is because the treatment of the tissue can often lead to fragmentation and/or degradation of the nucleic acid. However, the methods using the preamplification can be performed on very small fragments.

Allele Specific Methylation Methods

The methods can be used to analyze allele-specific methylation patterns. For example if there are different alleles. If there are two copies of each gene, e.g., the paternal copy is methylated and the maternal copy is not, the method can be used to determine the methylation pattern. In some embodiments, the method can be used to identify the number of methylations (multiple methylations per copy) and differences in methylation between the maternal and paternal gene. Thus, there are at least two applications: (1) identification of methylation of maternal vs paternal chromosomes, and (2) identification of multiple methylation sites on a single chromosome.

Both applications can use bisulfate conversion of genomic DNA which changes methylated cytosine into uracil. In standard methylation studies, TaqMan® SNP assays are used against the hypothesized methylated site to determine whether it contains a C or T. If the site has a C, then the site was not changed by bissulite treatment and therefore must not have been methylated. If the site has a T, then it must have been converted to U by bisulfite treatment, and subsequently to T during PCR, therefore it must have been methylated. However, other assays are in development to identify this change and there are other commercially available assays to look at these sites, so any type of PCR-based assay that is appropriate can be used.

The maternal vs paternal test can be performed by looking for physical linkage between the SNP site bases that are known to be paternal or maternal and sites that are hypothesized to be methylated. For instance, given two genomic sites 10 kb apart: genomic site 1 known to be A/A in the father and G/G in the mother, and genomic site 2 which is hypothesized to be a methylated cytosine. The multiplex-digital PCR method can be used to look for instances where single strands of DNA are shown to have: site 1=A and site 2=C (paternal unmethylated), site 1=A and site 2=T (paternal methylated), site 1=G and site 2=C (maternal unmethylated), site 1=G and site 3=T (maternal methylated). In some embodiments, this can be done as a single step reaction with 4-dyes, or as a two-step reaction with a PreAmp step.

The linkage of multiple methylated sites can be performed by using multiple assays against multiple linked methylation sites. For instance, given a hypothesis that there are 10 cytosines which are methylated across 15 kb, TaqMan® SNP assays can be used that differentiate C vs T for all sites and test the bisulfite treated DNA. The data can be analyzed just like the linkage of polymorphic sites. This can be done in a two-step protocol using a PreAmp reaction with a mix of all 10 assays.

Single Cell Association Methods

The methods can be used for single cell association studies where intact cells are subject to limiting dilution and the physical linkage between the targets being tested is created by the physical structure of the cell itself. For instance, the method can be used for any two or more nucleic acids prevented from dilution away from each other by the plasma membrane (e.g., plasmids or lysogens). Thus, the methods can be used to identify the presence of a lysogen and the percentage of cells containing the lysogen. This is distinct from a measurement of the overall number of lysogens in a sample, which might result from either a uniform distribution of lysogens in all cells in the culture, or a high number of lysogens in a small percentage of cells and an absence of lysogens in the remainder of cells. The method for this example can use a multiplex of two classes of assays. For example, the first type can be PCR-based assays which detect targets that are present in every bacterial cell—for instance assays which detect bacterial genomic DNA. The second type can be assays that detect the target lysogen. A limiting dilution of intact bacteria can be used to run a set of reactions which contain one or fewer bacterial cells as determined by amplification of the assays which detect bacterial genomic DNA. Detection of amplification of the assays which target the lysogen in the same reactions which contain genomic DNA is indicative of individual cells which contained that lysogen. In this example, detection of amplification of assays which target genomic DNA and failure of amplification of assays targeting the lysogen is indicative of individual cells which do not contain the lysogen. The percentage of cells containing the lysogen in the original sample can be determined by the ratio of cells that are positive for both genomic DNA targets and lysogen targets to the total number of detected cells.

The method can also be used to analyze a population of cells in the process of differentiation to see which genes (associated with determination, differentiation or disease) are turned on in which cells. The method can be used to determine which cells have differentiation gene(s) turned on and which have non-differentiation gene(s) turned on. Any type of differentiation can be analyzed, including but not limited to; neuronal, muscular, hematopoietic, epithelial, or bone differentiation. The cells can be analyzed to determine if they are mesenchymal, hematopoietic, or epithelial stem cells. The cells can be analyzed to determine if they are multipotent stem cells. The method for this example can use a multiplex of assays to the mRNA of genes associated with these states. The read-out of these assays can be simple on/off for presence/absence, or can be quantitative to measure low to high expression in individual cells.

Full Alternate Splicing Mapping Methods

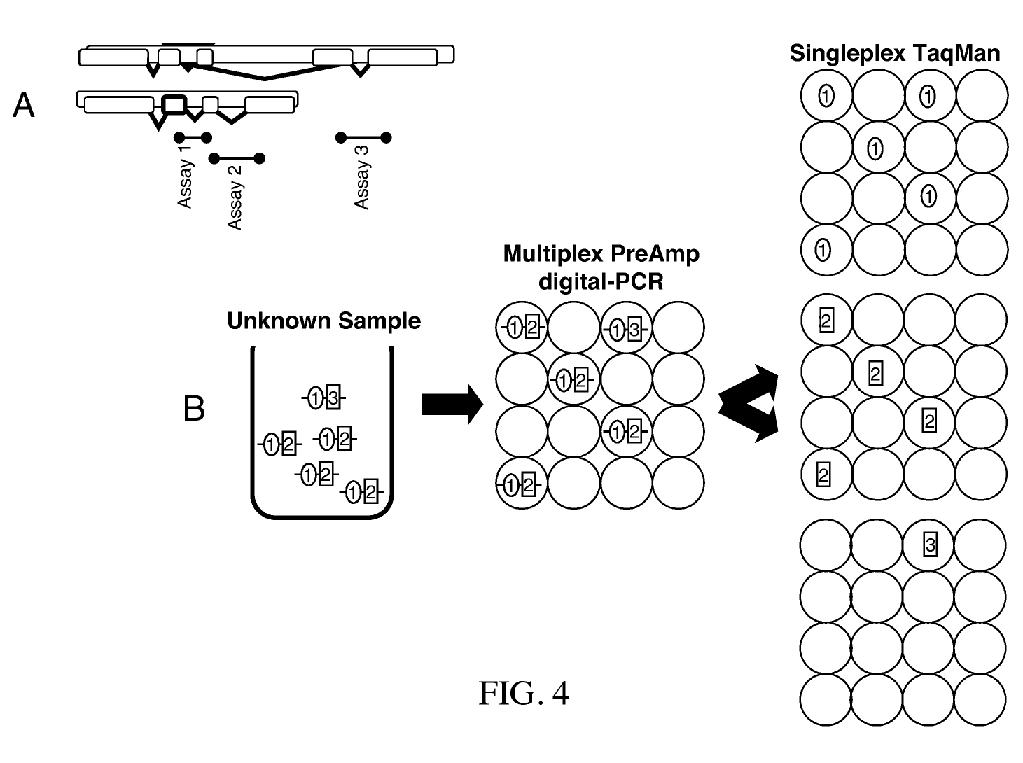

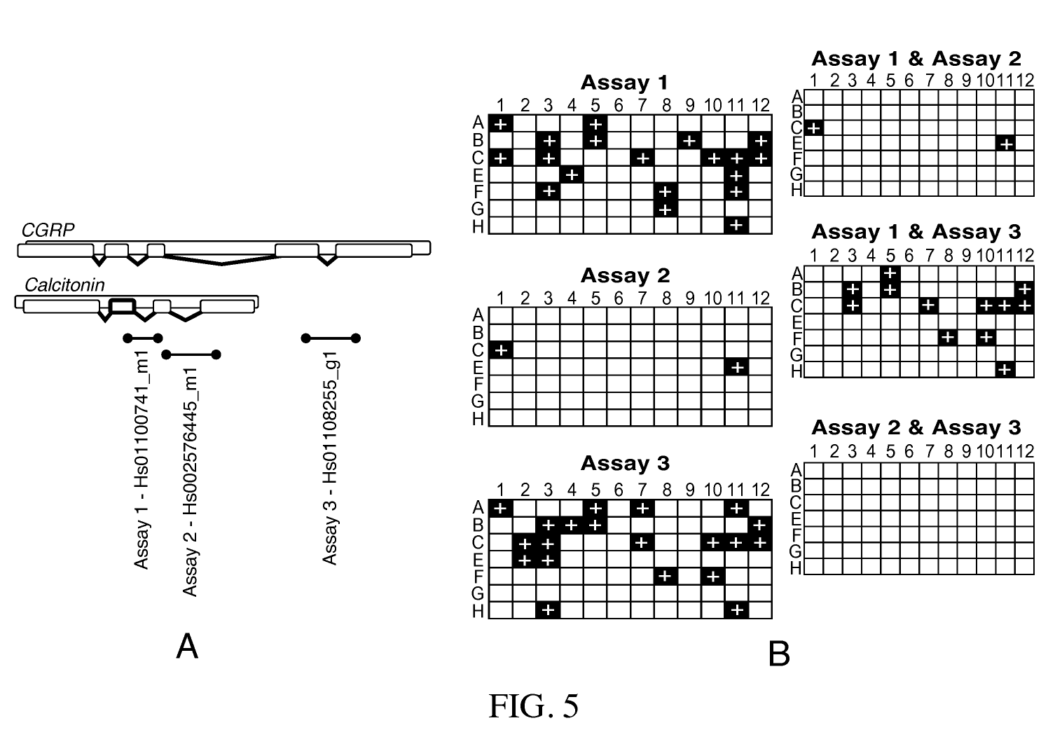

The methods can be used to identify and quantitate alternative splicing in a sample. It is not usually possible to use quantitative PCR to detect the number of different splice variants in a particular sample. For example, FIG. 5 shows a gene with 7 exons, the Calcitonin-related polypeptide alpha gene. This gene has two splice variants: CGRP and the Calcitonin. Quantitative PCR may be able to detect that there is more than one splice variant, but it will usually not be able to detect how much of each isoform there is (e.g., 20% of isoform A and 80% of isoform B). However, the methods identifying linkage as taught herein, can be used to identify the number of isoforms and/or the amount of each isoform. The methods can involve multiplexing all of the isoforms in the first step and then looking at each in the second step to find out how many isoforms the daughters had by whether there is linkage between the exons. FIG. 4 shows a method for identifying and quantifying mRNA splice forms in an uncharacterized sample. FIG. 4A shows a gene with two known mRNA isoforms with alternate 5′ domains. In this example, assays which target the exon-exon junctions of the mature mRNA are selected. Assay 1 is present in both isoforms. Assay 2 is present in only one isoform. Assay 3 is present only in the second isoform. In FIG. 4B, a sample of cDNA with unknown ratios of isoform 1 and isoform 2 is used as source material for a digital PCR reaction using a multiplex pool of all three assays (Multiplex PreAmplification). In this example, no detection is required in the PreAmplification reaction. Aliquots from the PreAmplification product are used as template in individual singleplex reactions to each of the three assays. Detection of amplification in the singleplex reactions is compared for all targets. Overlap of detection indicates that a cDNA molecule containing both exon junctions was present in the PreAmplification reactions and in the original sample. Digital counting can be used to calculate the ratios of isoforms present in the original sample.

The general principles of multiplex digital PCR can be expanded to a large number of additional applications where the physical linkage between assayable targets is biologically relevant. These include, but are not limited to high resolution genetic linkage mapping, allele specific methylation studies, single cell association studies, and full alternate splicing mapping.

Having described the invention with a degree of particularity, examples will now be provided. The following examples are intended to illustrate but not limit the invention.

### EXAMPLES

The following examples provide methods and compositions for identifying physical linkage for targets using multiplex Digital PCR. The targets may be polymorphic or non-polymorphic.

Materials and Methods:

The following methods were used for all of the experiments detailed below in the Examples except as otherwise noted.

Oligonucleotide sequences—synthesis and modification. The oligonucleotides were chemically synthesized using standard phosphoramidite-based nucleoside monomers and established solid phase oligomerization cycles according to Beaucage, S. L. and Iyer, R. P. (Tetrahedron, 1993 (49) 6123; Tetrahedron, 1992 (48) 2223). RNA phosphoramidites were protected with 2′O-TBDMS groups. Synthesis of oligonucleotides was performed on a BioAutomation MerMade 192 or BioAutomation MerMade 12 synthesizers (BioAutomation Corp, Plano, Tex.). Eight equivalents of activator were used for every equivalent of phosphoramidite to provide a satisfactory stepwise coupling yield of >98% per base addition. Purification of the individual oligonucleotides for in vitro screening was carried out using high throughput desalting and alcohol precipitation techniques. Analytical HPLC (ion exchange and/or reverse-phase) was used for determining single strand purity, MALDI mass spectrometry was used for determining oligonucleotide identity, and UV spectroscopy was used for quantitative determination of inhibitors.

Limiting Dilution—Samples were diluted to contain approximately 1 target per four wells.

The TaqMan® PreAmp Master Mix Kit (Life Technologies) was used in the following examples. The kit provides the reagents and protocols necessary to amplify a sample of nucleic acids. The TaqMan® PreAmp Master Mix Kit is intended for use with very small quantities of cDNA (1 to 250 ng). This kit allows one to increase the quantity of specific cDNA targets for gene expression analysis using TaqMan® Gene Expression Assays. Starting material is increased prior to PCR and the resulting preamplification product is then used for PCR.

### Example 1

### Multiplex Digital PCR of 20 Targets

An experiment was performed to assess the ability of multiplex-digital PCR as a method to detect known physical linkage of genomic domains. A diagram of the general method of multiplex PCR is shown in FIG. 2.

20 TaqMan® SNP Assays from the Drug Metabolizing Enzymes set ABCC1, ABCB1, and ABCB4 genes were selected. The SNPs were known to be located in three clusters on chromosomes 7 and 16 of <14 kb each. The relative position of each assay in the cluster was indicated in bases from the location of the first assay. The three domains were associated with the ABCC1, ABCB1, and ABCB4 genes. The clusters associated with ABCB1 and ABCB4 neighbor each other and cover a domain of 75 kb. Neither the allelic status of the assay targets, the haloptype, nor the biological activity of the genes was assessed as part of this experiment.

A single donor human DNA sample was diluted to 0.8 pg/μl (0.25 genomes/μl) in TE and a TaqMan®PreAmp pool containing all 20 assays at a concentration of 0.2× each was constructed. Using the PreAmp pool, 32 independent multiplex TaqMan® PreAmp reactions were run on 1 μl DNA samples (PreAmp wells 11A to 12P), and 16 independent No Template Control (NTC) multiplex PreAmp reactions were run on 1 μl of TE (NTC wells A to P). TaqMan® PreAmp reactions contained: 1 μl DNA or TE, 2.5 μl 2× TaqMan® PreAmp Master Mix, 1.25 μl PreAmp Pool, 0.23 μl H₂O. Reactions were run for 15 cycles using standard PreAmp conditions as indicated in the TaqMan®PreAmp Master Mix manual (Life Technologies). Reactions were run on the Applied Biosystem® 9600. The PreAmp product was diluted 1:8 with TE.

For each multiplex PreAmp reaction, 20 singleplex TaqMan® reactions were run corresponding to each of the 20 SNP assays. Singleplex reactions contained: 2.5 μl TaqMan® Genotyping Master Mix, 0.25 μl 20× Assay, 1.25 μl dilute PreAmp Product, and 1 μl H₂O. Reactions were run for 50 cycles using standard thermal conditions as indicated in the TaqMan® Genotyping Master Mix manual. Reactions were run in real-time mode on the Applied Biosystems® 7900 instrument.

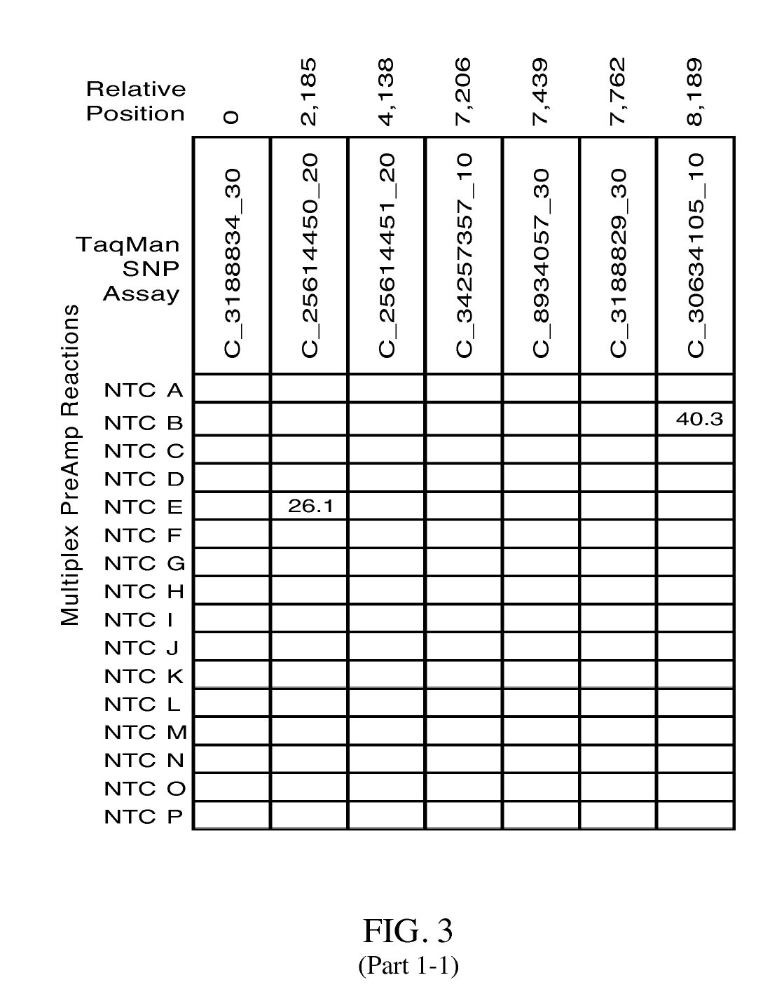

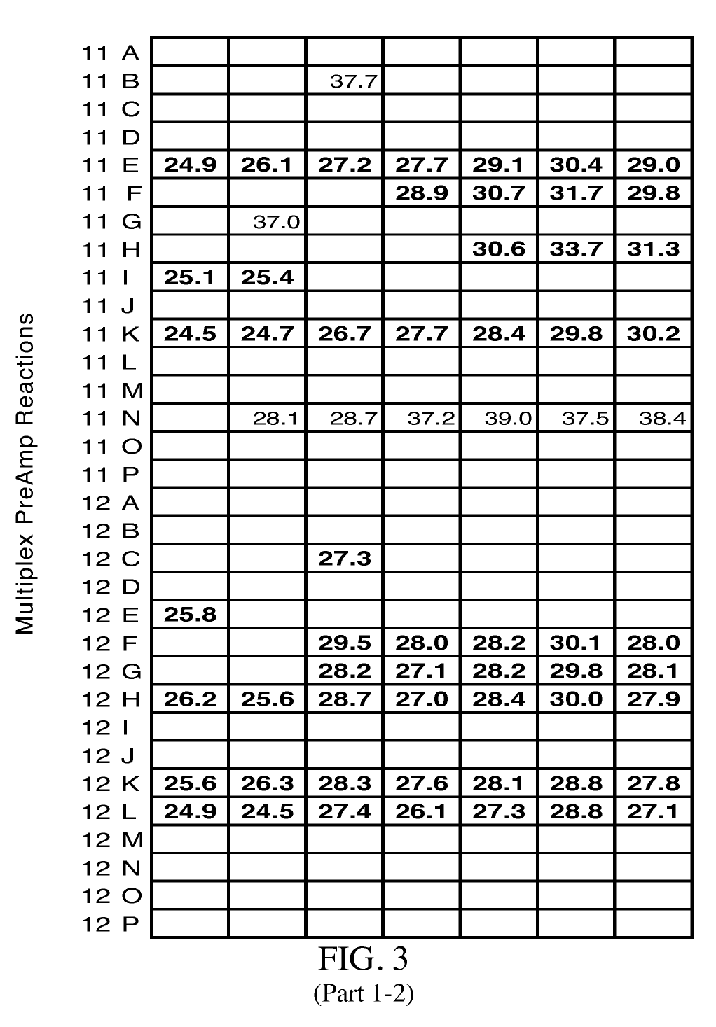

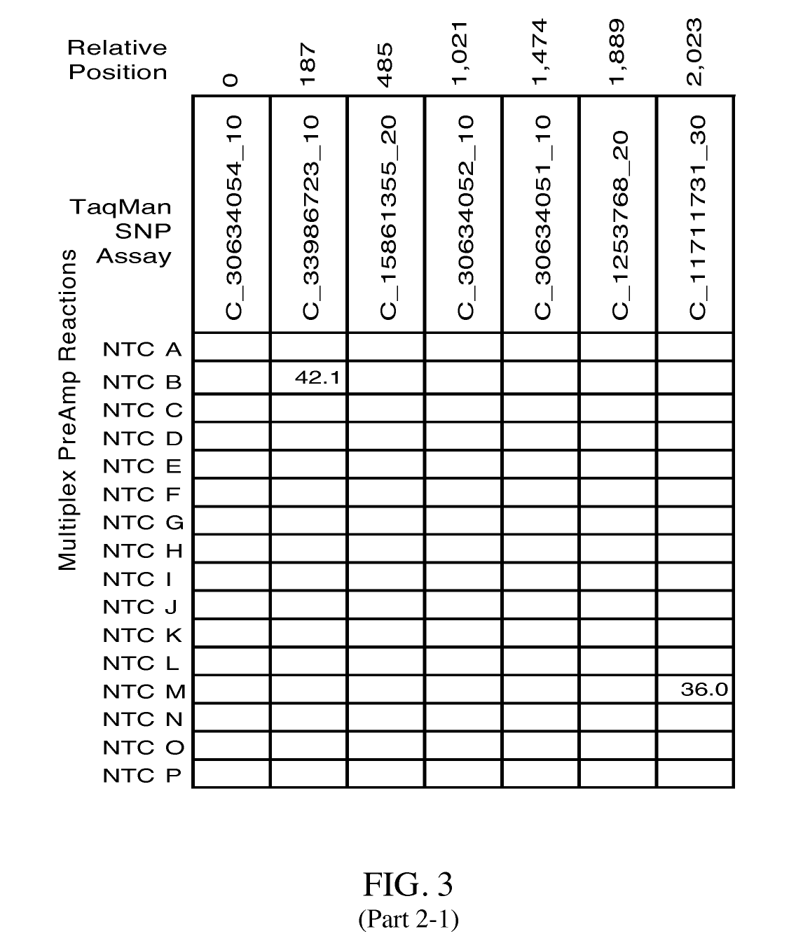

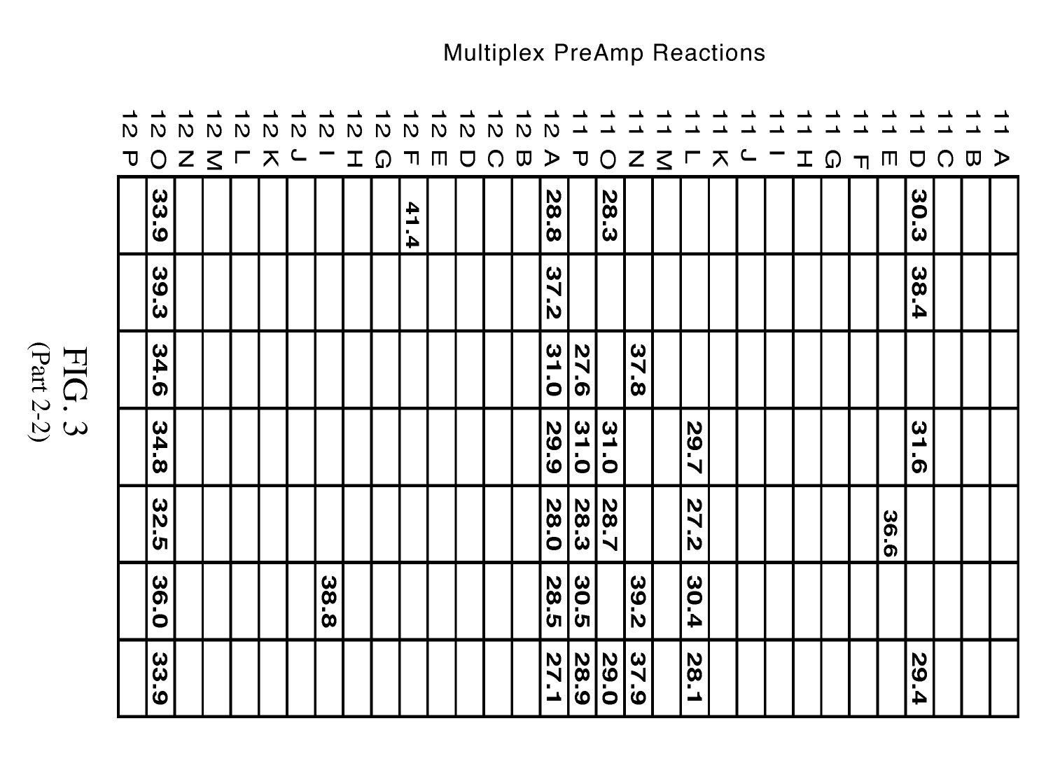

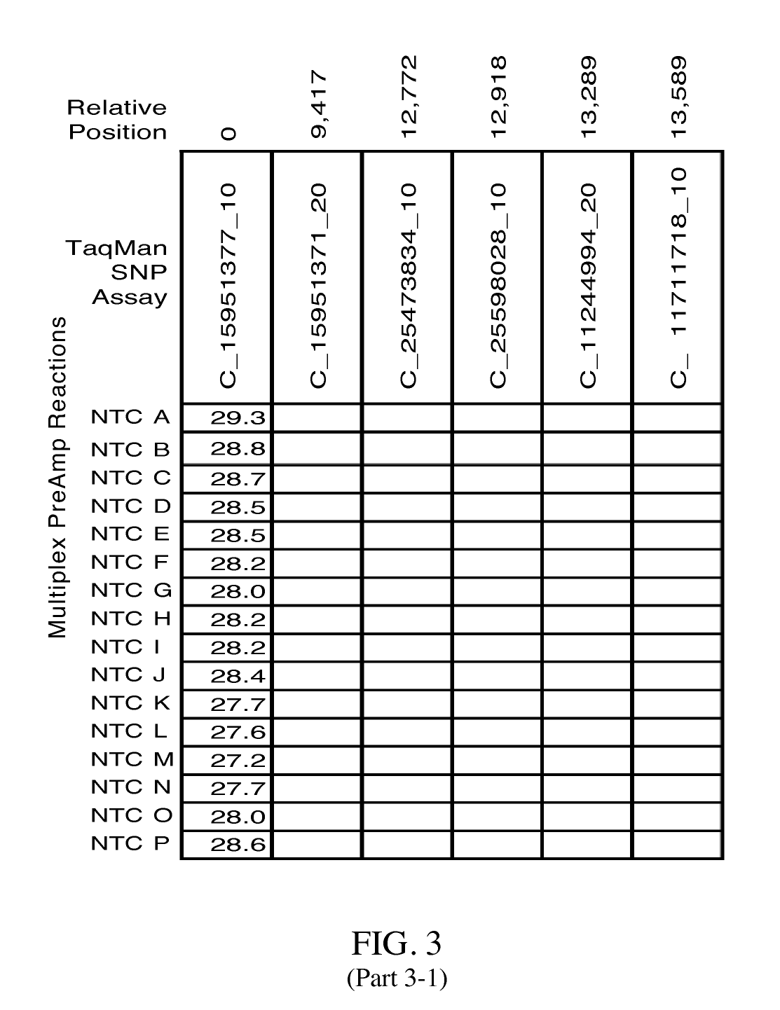

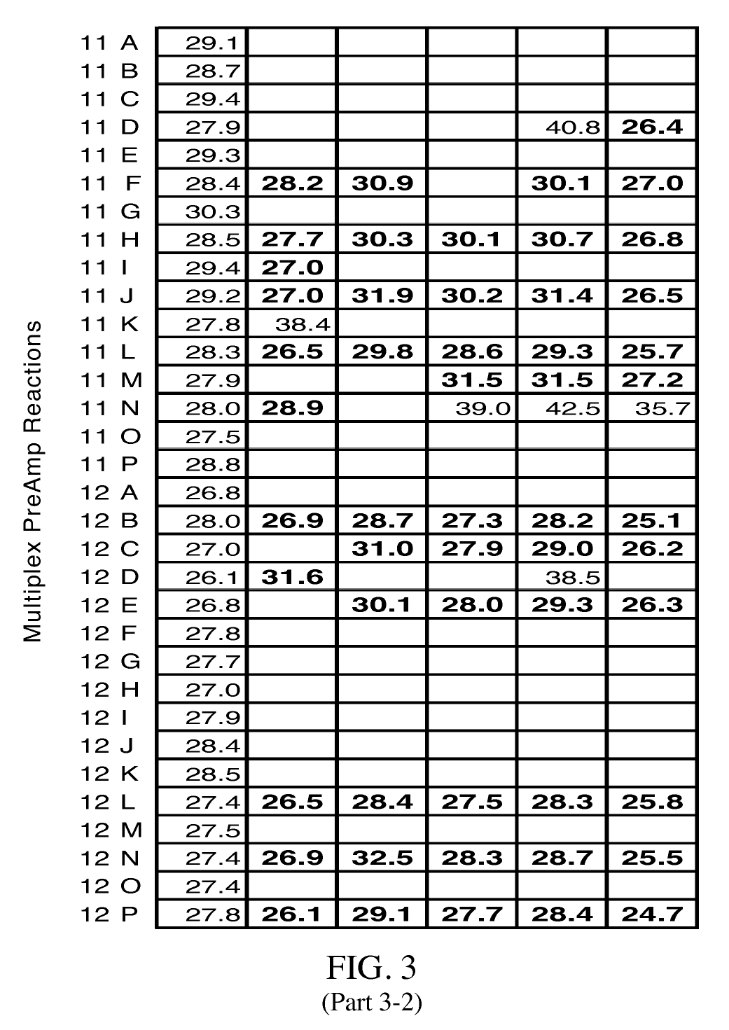

In FIG. 3, the CTs are shown for each singleplex reaction, allele specificity was not assessed. The assays are arranged in FIG. 3 in columns and correspond to the ordering of the targets on the chromosome. The source PreAmp reactions were arranged in rows. The presence of rows of continuous positive reactions indicated targets which amplified in multiple singleplex reactions from the same PreAmp reaction. This indicates that single DNA molecules with physically linked targets were present in the PreAmp reaction, and thus were physically linked in the source DNA. Linkage of targets within the three clusters was evident, although there was no linkage observed between the three clusters themselves.

The assay C_{—}15951377_{—}10 gave positive amplification in all NTC and experimental reactions and was thus uninformative as to the linkage state of this target. This showed that the input of 0.25 genomes (0.8 pg) was appropriate for digital PCR with 4 out of 16 reactions showing amplification. Similar results were observed with 5 different single assays (e.g., 4, 2, 2, 6, 4 assays out of 16).

### Example 2

### Multiplex-Digital PCR as a Method to Detect and Quantify mRNA Isoforms

In this example a gene with two known mRNA isoforms with alternate 5′ domains was tested to identify splice forms. FIG. 4A provides a map showing the isoforms and an assay which targets the exon-exon junctions of the mature mRNA selected to identify the presence of the isoforms. Assay 1 is present in both isoforms and Assay 2 is present in only one isoform.

Multiplex PreAmp digital PCR was performed on the mRNA diluted to limiting dilution using the method shown in FIG. 4B. A sample of cDNA with unknown ratios of isoform 1 and isoform 2 was used as source material for a digital PCR reaction using a multiplex pool of all three assays (Multiplex PreAmplification). No detection was required in the PreAmplification reaction. Aliquots from the PreAmplification product were used as template in individual singleplex reactions to each of the three assays (see FIG. 4B). Detection of amplification in the singleplex reactions was compared for all targets. Overlap of detection indicates that a cDNA molecule containing both exon junctions was present in the PreAmplification reactions and in the original sample. Digital counting was used to calculate the ratios of isoforms present in the original sample.

In FIG. 5, the assay was performed for the Calcitonin-related polypeptide alpha. As shown in FIG. 5A, Calcitonin-Related Polypeptide Alpha was known to express two different mRNA isoforms. In human hypothalymus, the CGRP isoform is in greater abundance than the Calcitonin isoform (Rosenfeld, et al., PNAS-USA, 79:1717 (1982); Amara, et al., Nature, 298:240 (1982)). As shown in FIG. 5A, three TaqMan® gene expression assays were selected to target exon-exon junctions in the mature mRNA isoforms. Assay 1 (Hs01100741_m1) targeted both isoforms. Assay 2 (Hs002576445_m1) and Assay 3 (Hs01108255_g1) targeted alternate isoforms.

A preamplification pool containing all forward and reverse primers for Assays 1, 2, and 3 was used in the 1st round of a multiplex digital-PCR. 84 reactions were run on 1st strand cDNAs from human hypothalamus. The PreAmp PCR and qPCR were performed as in Example 1 according to the manufacturer's instructions. Three individual 2nd round reactions were run on each 1st round product. Coincident localization assays refer to coincident amplification which is the amplification of two or more targets occurring simultaneously in the same physical well. As shown in FIG. 5B, the signals for the Coincident Localization Assays 1 and 2 confirmed the tissue specific expression of the first (Calcitonin) isoform in the hypothalamus. The signals for the Coincident Localization of Assays 1 and 3 confirmed the tissue specific expression of the first (CGRP) isoform in the hypothalamus. Based on the frequency of positives with Assay 1 (22.2%) and Assay 2 (23.8%), the expectation for overlap due to random chance was 5.3%. The observed overlap was 14.2%. 63.1% of reactions with Assay 1 signal also had Assay 3 signal. The results of the multiplex digital-PCR confirmed the known isoform relationships of Calcitonin-Related Polypeptide Alpha in human hypothalymus.

Each embodiment disclosed herein may be used or otherwise combined with any of the other embodiments disclosed. Any element of any embodiment may be used in any embodiment.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

1. A method of identifying physical linkage between two or more nucleic acid targets in a sample, the method comprising:
a) diluting the sample via limiting dilution;
b) aliquoting the diluted sample into wells;
c) performing multiplex PCR in each chamber, wherein a first dye is used for the first target and a second dye is used for the second target; and
d) identifying the presence of the first and second dyes in the wells, wherein a non-random distribution of the dyes identifies the targets as linked.

2. The method of claim 1, wherein the dye is attached to a probe.

3. The method of claim 1, wherein the dye is attached to a primer.

4. The method of claim 1, wherein the multiplex PCR employs FRET, TaqMan®, Molecular Beacon, Amplifluor®, Scorpion™, Plexor™, and/or BHQplus™.

5. The method of claim 1, wherein the non-random distribution is determined by Student's T-test, F-test, Chi-square test, Fisher-test, ANOVA test, and/or multiple comparison ANOVA test.

6. The method of claim 1, wherein the non-random distribution is determined by a p-value of <0.05.

7. The method of claim 1, wherein the targets are chosen from: SNPs, InDels, mutations, translocations, inversions, duplications, deletions, ring chromosomes, isochromosomes, splice regions, microsatellites, mature microRNAs, pri-microRNAs, pre-microRNAs, non-coding RNAs, mRNAs primary transcripts, genomic loci, alleles, multi-RNA complexes, splice variants, transposons, ribozymes, bacterial genes, viral nucleic acids, ribosomal RNAs, viral insertion sites, vector insertion sites, hypervariable regions, mitochondrial DNA, highly polymorphic regions, MHC regions, and MHC gene products.

8. The method of claim 1, wherein one or more of the targets is polymorphic.

9. The method of claim 1, further comprising preamplification of the sample before diluting the sample.

10. The method of claim 1, further comprising determining the original linkage distance of the two targets.

11. The method of claim 1, further comprising at least a third target and a third dye.

12. The method of claim 1, further comprising at least four or more targets and four or more different dyes.

13. The method of claim 1, wherein the dye is a fluorescent dye or a quantum dot.

14. The method of claim 1, wherein the dye is chosen from: FAM, TET, HEX, Cy3, TMR, ROX, Texas red, LC red 640, Cy5, VIC, SYBR, LC red 705, JUN, ABY, TED, TZA, and SID.

15. The method of claim 1, wherein the method is used to determine the linkage between one or more alleles of a polymorphic site.

16. The method of claim 1, wherein the method is used to determine the linkage distance between one or more alleles of a polymorphic site.

17. The method of claim 1, wherein the method is used to determine haplotype or haplogroup.

18. The method of claim 1, wherein the method is used for DNA profiling, DNA testing, DNA typing genetic fingerprinting, ancestry analysis, paternity testing, and/or maternity testing.

19. The method of claim 1, wherein the method is used for disease association, disease risk assessment, patient stratification, drug metabolism analysis and/or diagnosis.

20. The method of claim 1, wherein the amplification product is identified by sequencing, partial sequencing and/or hybridization.

21-62. (canceled)
